(19) United States
(12) Patent Application Publication
(10) Pub. No.: US 2012/0171682 A1
(43) Pub. Date: Jul. 5, 2012

(54) METHOD FOR DETECTING, IDENTIFYING AND/OR QUANTIFYING CARBON NANOTUBES

(75) Inventors: Gilles Marchand, Pierre-Chatel (FR); Dorothee Jary, Sassenage (FR); Pierre Puget, Saint Ismier (FR); Francois Tardif, Lans En Vercors (FR)

(73) Assignee: COMMISSARIAT A L'ENERGIE ATOMIQUE ET AUX ENE ALT, Paris (FR)

(21) Appl. No.: 13/255,387

(22) PCT Filed: Mar. 8, 2010
(86) PCT No.: PCT/EP2010/052921

§ 371 Date: Nov. 21, 2011

(30) Foreign Application Priority Data
Mar. 9, 2009 (FR) 0951461

(51) Int. Cl. C12Q 1/68; B82Y 35/00

(52) U.S. Cl. 435/6.12; 977/742; 977/842

(57) ABSTRACT

The present invention relates to a method and a kit for detecting, optionally identifying and optionally quantifying at least one carbon nanotube possibly included in a sample, including the steps consisting in: (a) subjecting said sample to conditions enabling the amplification of a nucleotide sequence using primers capable of amplifying said nucleotide sequence, the possibly included carbon nanotube having been functionalized by said nucleotide sequence prior to step (a), and (b) detecting, optionally identifying and optionally quantifying the amplification product possibly obtained after step (a).

### TECHNICAL FIELD

The present invention belongs to the field of nanotechnologies and, in particular, the field of nano-objects such as carbon nanotubes.

Indeed, the present invention aims to provide a method making it possible to detect, identify and/or quantify carbon nanotubes. The method according to the invention is based on the functionalization of the nanotubes by a nucleotide sequence and on a biological amplification to which the nanotubes thus functionalized are subjected. The method according to the invention makes it possible to obtain a very low nanotube detection limit and is easy to implement in the workplace and to monitor the environment.

The present invention also proposes a kit for carrying out such a method.

### PRIOR ART

Owing to their exceptional properties, carbon nanotubes (CNTs) are much-studied nanomaterials at this time. Thousands of scientific publications involving CNTs come out each year.

Let us first recall that a carbon nanotube is defined as a concentric coil or one or more layers of graphene (carbon hexagonal tiling). The term “Single Wall NanoTube” (SWNT) is used when a single layer of graphene is used, and “Multi Wall NanoTube” (MWNT) is used in the case of several layers of graphene. Due to their unique structure and their dimensions characterized by a high length/diameter ratio, nanotubes have exceptional mechanical, electric and thermal properties.

Owing to these properties, an increasing number of applications using CNTs are being transferred from the laboratory to a commercial product. Thus, CNTs can already be found in tennis rackets, bicycles, TV screens and tires, as well as resins used by the aerospace industry, defense, microelectronics, and others.

CNT production will doubtless increase significantly in the coming years. For example, Bayer, one of the main CNT suppliers, announced a production capacity of 3000 t of CNTs per year in 2012. In this context, exposure to CNTs, especially for laboratory researchers and industrial workers, will increase considerably in the coming years. CNTs could also certainly be disseminated in the environment, therefore with potential risks for the human population as well as animals and/or plants. In fact, currently, an intense debate exists regarding the toxicity of CNTs: some consider that CNTs are not toxic [1,2], while others consider that they are [3-5].

It has therefore become important to develop a technique for detecting, quantifying and identifying CNTs in the workplace and in the environment. CNT detection strictly speaking is a very underdeveloped field that has been the subject of very few studies. Indeed, because of their nanometric dimensions, CNTs are very difficult to detect using traditional techniques. Thus, for example, they cannot be detected by phase contrast optical microscopy, a standard fiber detection technique [6]. However, scientists have proposed detecting CNTs using other microscopic or spectroscopic methods [7].

Microscopic techniques, such as Scanning Tunneling Microscopy (STM), Transmission Electron Microscopy (TEM), Scanning Electron Microscopy (SEM) or Atomic Force Microscopy (AFM), are techniques that can be used to detect CNTs. However, these techniques have a relatively good sensitivity and selectivity. But the usage conditions for these techniques are very critical for routine analyses. In particular, this requires that one already know with precision the location of the carbon nanotubes, the aforementioned techniques only being effective upon confirmation of the presence of said CNT. They also cannot be carried out with portable devices. Lastly, they do not allow identification of the nanotubes, which can be bothersome in case of incident, but also to manage a production method.

Spectroscopic techniques, such as Raman and fluorescence spectroscopies, can be adapted to routine analyses. However, Raman spectroscopy only shows selectivity for certain CNTs, in particular SWNTs. Weisman et al. established a method for detecting SWNTs by fluorescence in intact organisms [8]. This sensitive and selective method only makes it possible to detect individual SWNTs (i.e. monodispersed). Furthermore, the need to integrate lasers with different wavelengths into a device for detecting, identifying and possibly quantifying CNTs, in particular during production methods, increases the cost of said device and the CNTs. Lastly, spectroscopic techniques do not make any discrimination possible between the different CNTs.

There is therefore a real need for a method making it possible to detect, possibly identify and possibly quantify one or more CNTs with great selectivity and sensitivity.

### DESCRIPTION OF THE INVENTION

The present invention makes it possible to resolve the technical problems and drawbacks previously listed. In fact, the inventors took an interest in the detection of CNTs using the traditional biochemical techniques such as amplification techniques following the functionalization of the CNTs by nucleotide sequences.

This amplification makes it possible to achieve very low detection limits. It also makes it possible to perform real-time or end-of-reaction detection. It also makes it possible to quantify the CNTs.

Additionally, the functionalization of the CNTs by nucleotide sequences allows these CNTs to be identified. In fact, the nucleotide sequences are primarily made up of nucleotides (A, T, C, G, U or others) that can be associated in an infinity of positions thereby forming a primary sequence that can be likened to a bar code.

As a result, the present invention relates to a method for detecting, possibly identifying and possibly quantifying at least one carbon nanotube that may be present in a sample comprising the following steps consisting in:

(a) subjecting said sample to conditions enabling the amplification of a nucleotide sequence using primers capable of amplifying said nucleotide sequence,

the possibly included carbon nanotube having been functionalized by said nucleotide sequence prior to step (a), and

(b) detecting, optionally identifying and optionally quantifying the amplification product possibly obtained after step (a).

In a first embodiment of the present invention, the carbon nanotube that the sample can contain has been functionalized by a nucleotide sequence prior to taking of a sample that may contain such a carbon nanotube, and in particular during the method for preparing said carbon nanotube. Thus, the method according to the present invention advantageously comprises the following successive steps consisting in:

a₁) preparing at least one carbon nanotube functionalized by at least one nucleotide sequence;

b₁) taking a sample that may contain said carbon nanotube;

c₁) subjecting said sample to conditions enabling the amplification of said nucleotide sequence using primers capable of amplifying said nucleotide sequence; and

d₁) detecting, optionally identifying and optionally quantifying the amplification product possibly obtained after step (c₁).

In this embodiment, the functionalization makes it possible to detect and identify the carbon nanotube present in a sample. In fact, during the preparation of said nanotube, its functionalization by a particular nucleotide sequence enables the traceability of said nanotube. This embodiment is particularly adapted to quality control and verification of the CNT production. It is possible to consider that, during preparation thereof, different batches of CNTs distributed as a function of criteria such as their nature, production site, preparation method, toxicity class, elimination procedure to be followed, any chemical modification, their number of walls, average length, average width are marked by particular nucleotide sequences specific to each batch.

Thus, during implementation of the method according to the invention, a mixture of primers capable of amplifying said nucleotide sequences can be used during step (b₁) so as to precisely identify the nanotubes present in the sample.

In a second embodiment of the present invention, the carbon nanotube that the sample can contain is functionalized by a nucleotide sequence after taking said sample. Thus, in this embodiment, the method according to the present invention advantageously comprises the following successive steps consisting in:

a₂) putting said sample in contact with at least one nucleotide sequence under conditions enabling the functionalization of the nanotube(s) that may be present by said nucleotide sequence;

b₂) eliminating any nucleotide sequence not involved in the functionalization;

c₂) subjecting said sample to conditions enabling the amplification of said nucleotide sequence using primers capable of amplifying said nucleotide sequence; and

d₂) detecting and optionally quantifying the amplification product that may be obtained after step (c₂).

All of the following definitions and alternatives apply to the method according to the present invention and in particular to these two embodiments, unless otherwise explicitly indicated.

In the context of the present invention, the sample used can be any sample, liquid or solid, which may contain or be contaminated by one (or more) carbon nanotube(s). Advantageously, said sample is chosen from the group consisting of a biological sample; a sample of city, river, sea, lake, ground or air-cooled tower water; aerial sample; ground sample; a sample obtained on an industrial site; or a mixture thereof.

“Biological sample” refers, in the context of the present invention, to a sample obtained from a vegetable or animal organism such as a human, living or dead. This biological sample can in particular be a sample obtained from a whole plant or part of a plant such as the stem, roots, flowers, leaves, seeds or sap. This biological sample can also be any fluid naturally secreted or excreted from a human or animal body or any recovered fluid, from a human or animal body, such as blood, blood serum, blood plasma, lymph, saliva, sputum, tears, sweat, sperm, urine, stool, milk, cerebrospinal fluid, interstitial liquid, an isolated bone marrow fluid, a mucus or fluid from the respiratory, intestinal or genito-urinary tract, cell extracts, tissue extracts and organ extracts.

“Sample obtained on an industrial site” refers, in the context of the present invention, to a sample obtained on any industrial site and, in particular, a site on which carbon nanotubes are produced, packaged and/or stored or an industrial waste site. This sample can assume the form of dust in particular recovered on the ground, on any production device or any aeration filter.

The sample used in the context of the present invention can have a highly variable volume and can have been obtained using any technique known by those skilled in the art such as extraction, suction, sampling or washing.

Furthermore, before carrying out the method according to the invention, said sample can undergo a preparatory treatment such as enzyme treatment, shredding, dilution, solubilization, centrifugation and/or filtration. One preferred enzyme treatment consists of subjecting said sample to the action of at least one nuclease such as an RNAse, a DNAse and/or an endonuclease in order to eliminate any nucleotide sequence present in the sample and likely to yield “false” positives, with the understanding that the enzyme used is inactive relative to the end by which the nucleotide sequence is attached to the CNT. This treatment using one (or more) nuclease(s) is particularly adapted for the second embodiment of the inventive method.

In the context of the second embodiment of the present invention, said sample advantageously undergoes a preparatory treatment aiming to isolate and/or purify any CNTs present, before their functionalization, i.e. before step (a₂) of the method. This preparatory treatment is used to eliminate any element other than a CNT present in the sample and that can be functionalized by the nucleotide sequence and yield “false” positives. Such a preparatory treatment is well known by those skilled in the art and consists of traditional purification, such as centrifugation.

The expression “nucleotide sequence” used in the present document is equivalent to the following terms and expressions: “nucleic acid,” “polynucleotide,” “nucleotide molecule,” “polynucleotide sequence.” The nucleotide sequence in the context of the present invention is advantageously chosen from the group consisting of oligonucleotide, possibly modified; a desoxyribonucleic acid (DNA), possibly modified, such as single-helix or double-helix, genomic, chromosomal, chloroplastic, plasmidic, mitochondrial, recombinant or complementary DNA; a ribonucleic acid (RNA), possibly modified, such as a messenger, ribosomal, transfer RNA; a portion and a fragment thereof.

“Modified oligonucleotide” (or DNA or RNA) refers, in the context of the present invention, to an oligonucleotide (or a DNA or RNA), natural or synthetic, chemically modified in particular to increase the effectiveness and selectivity during the functionalization of the CNTs. Thus, examples of chemical modifications include the introduction of an amine or a thiol to the 5′-terminal end of the nucleotide sequence. With these functions, the nucleotide sequence can react with activated carboxylic acids [9] or maleimides [10] that have been grafted on the CNTs. A covalent bond is thus formed between the carbon nanotube and the nucleotide sequence.

The nucleotide sequence that can be used in the context of the present invention can be extracted from living organisms such as animals, plants, yeasts, bacteria, fungi or chemically synthesized. The techniques making it possible to extract a nucleotide sequence from an organism, like the techniques making it possible to synthesize a nucleotide sequence, are well known by those skilled in the art.

Particularly advantageously, the nucleotide sequence used in the context of the inventive method is a non-natural synthetic sequence, in particular customized.

The nucleotide sequence that can be used in the context of the present invention advantageously comprises from 20 to 3,000 nucleotides, notably 20 to 1,000 nucleotides, in particular from 30 to 150 nucleotides and, more particularly, from 40 to 120 nucleotides.

In the context of the second embodiment of the inventive method, the nucleotide sequence can have an identical size or comprise, for example, advantageously from 20 to 100 nucleotides, in particular from 30 to 80 nucleotides and, more particularly, 40 to 60 nucleotides. Any genetic sequence can be used whether it is found in nature or not, since this embodiment provides for treatment of the sample intended to eliminate any nucleotide sequence present in the latter, before the functionalization in step (a₂) of the method.

Furthermore, it is possible to code information on this nucleotide sequence, in particular in the context of the first embodiment of the inventive method. In that case, said nucleotide sequence associates several blocks, each block comprising between 10 and 20 bases. The number of blocks can vary and the state of the art in oligonucleotide synthesis makes it possible to consider constructs comprising up to 2 outer blocks (hereafter B blocks) with 15 bases making up the sequences recognized by the primers during steps (a), (c₁) and (c₂) and 6 inner blocks with 15 bases, for a total length of 120 bases. The two B blocks can be generic, i.e. the same consensus sequences have been selected by all CNT producers. They are therefore identical irrespective of the origin of the CNTs and there will always be amplification in steps (a), (c₁) and (c₂), if CNTs are in fact present in the tested sample. They can also be specific and correspond a priori to rather general information such as the production site, each site being defined by a particular pair of primers.

The internal blocks mainly used during the detection steps and in particular for microarray hybridization or for hybridization of a specific probe used during the PCR in real-time, can have sequences corresponding to different information such as the toxicity class of the CNT, the elimination procedure to be followed, its possible chemical modification, number of walls, average length and average width.

The sequences used for all of the coding blocks advantageously do not correspond to any genetic sequence found in nature so as not to have “false” positives. If this is not possible, only the B blocks will be designed not to code for any sequence found in the living world or at least for species that can be present and amplified by PCR during routine tests, such as bacteria, viruses, pollens, etc. Thus, according to the invention, the nucleotide sequence present on the CNTs can be made up of blocks with different compositions and sequences.

The construction of the polynucleotides may also comprise a spacer sequence. “Spacer sequence” refers, in the context of the present invention, to a sequence of approximately ten bases that space the coding zone of the nucleotide sequence from the functionalized CNT. This spacer sequence makes it possible to decrease the steric bulk due to the CNT that can decrease the amplification output during steps (a), (c₁) and (c₂).

The present invention applies to all types of carbon nanotubes irrespective of how they are prepared. Thus, the carbon nanotubes to be detected, optionally to be identified and optionally to be quantified using the method according to the invention, can be nanotubes with a single layer of graphene (SWNT), nanotubes with multiple layers of graphene (MWNT), or a mixture of SWNT nanotubes and MWNT nanotubes. These nanotubes can have a length between 10 nm and 10 mm, in particular between 100 nm and 10 μm, in particular between 500 nm and 3 μm.

One skilled in the art knows the different techniques making it possible to prepare such carbon nanotubes. Examples include physical methods based on sublimation of the carbon such as electric arc methods, laser ablation methods, or using a solar oven and the chemical methods consisting of pyrolyzing carbonaceous sources on metal catalysts and similar to the chemical vapor deposition (CVD) method such as, in particular, the pyrolysis method covered by international application WO 2004/000727 [11].

The present invention requires the functionalization of the carbon nanotubes using one (or more) nucleotide sequence(s). As already explained, this functionalization can take place before the sample to be tested is taken (first embodiment of the inventive method) or after said sample is taken (second embodiment). The time interval separating steps (a₁) and (b₁) or the prior sample-taking and step (a₂) can be in the vicinity of several minutes, several hours, several weeks, or even several months.

The functionalization of the CNTs by biomolecules has been greatly studied for biological and biomedical applications. The functionalization methods of the CNTs can be divided into two categories: covalent functionalizations and non-covalent functionalizations such as physio-adsorptions.

As a result, in a first alternative, during functionalization of the CNT(s) by one (or more) nucleotide sequence(s) in the context of the method according to the invention and in particular in the context of steps (a₁) and (a₂), the nucleotide sequence covalently bonds to said nanotube(s). Likewise, several nucleotide sequences, identical or different, can covalently attach to a same CNT.

Advantageously, this covalent bond can be indirect. In fact, the functionalization of the CNTs by one (or more) nucleotide sequence(s) is done via a spacer. “Spacer” refers, in the context of the present invention, to a chemical compound that can covalently bind, on the one hand, to a CNT and, on the other hand, to a nucleotide sequence. Such a spacer therefore comprises two distinct chemical functions that are capable of forming a covalent bond, one with a group supported by the CNT and the other with a group supported by the nucleotide sequence. Advantageously, the covalent and indirect bond is done via a spacer having at least two distinct chemical functions, identical or different, chosen from the group consisting of a carboxyl function (capable of reacting with an amine or alcohol function), an aryl group (such as pyrene, naphthalene or polyaromatics), a radical entity, a hydroxyl function or an alcohol function (capable of reacting with a carboxyl or isocyanate function), an amine function (capable of reacting with an ester function), an ester function (capable of reacting with an amine function), an aldehyde function (capable of reacting with a hydrazide function), a hydrazide function (capable of reacting with an aldehyde function), a ketone function (capable of reacting with two alcohol functions, acetalization), an epoxy function (capable of reacting with an amine function), an isocyanate function (capable of reacting with a hydroxyl function) a maleimide function (capable of reacting with a thiol function, an amine function or a diene), a diene (capable of reacting with a maleimide function) and a thiol function (capable of reacting with a maleimide function or another thiol function).

“Aryl group” refers, in the context of the present invention, to an aromatic or heteroaromatic carbonaceous structure, possibly mono- or polysubstituted, from 3 to 30 carbon atoms, formed by one (or more) aromatic or heteroaromatic ring(s) each including 3 to 8 atoms, the heteroatom(s) being able to be N, O, P or S. The substituent(s) can contain one or more heteroatom(s), such as N, O, F, Cl, P, Si, Br or S as well as C₁-C₆ alkyl groups.

“Polyaromatic aryl” refers to an aryl as previously defined having 2 to 10 aromatic or heteroaromatic rings and in particular 2 to 5 aromatic or heteroaromatic rings.

The state of the art knows different spacers, commercially available, usable in the context of the present invention. Said spacer can be made up of an alkyl group having at least two distinct chemical functions, identical or different, as previously defined. “Alkyl group” refers, in the context of the present invention, to a linear, branched or cyclic alkyl group, possibly substituted, with 1 to 16 carbon atoms, in particular 1 to 12 carbon atoms, in particular, from 1 to 8 carbon atoms, more particularly, from 1 to 6 carbon atoms and, still more particularly, from 1 to 3 carbon atoms. The substituent(s) can contain one or more heteroatoms, such as N, O, F, Cl, P, Si, Br or S as well as C₁ to C₆ alkyl groups. Advantageously, the alkyl group used in the context of the present invention is a methyl group, an ethyl group, a propyl group, an isopropyl group or a cyclopropyl group.

Examples of spacers that can be used in the context of the present invention include in particular the spacers described in international application WO 03/053846 [12] p-azidobenzoyl hydrazide, N-ε-maleimidocaproic acid, sulfosuccinimidyl 4-(N-maleimidomethyl)-cyclohexane-1-carboxylate and N-(p-maleimidophenyl)-isocyanate.

Since CNTs are not very reactive, the covalent functionalization of the CNTs by biomolecules such as a nucleotide sequence is often done in two steps: the generation of reactive sites on the CNTs followed by the reaction of those sites with the biomolecules or spacers.

Thus, in the context of the inventive method, the functionalization step comprises two sub-steps consisting in:

(i) subjecting the sample or said nanotube(s) to conditions enabling at least one reactive entity to be formed on the surface of a carbon nanotube, then

(ii) putting said sample or said nanotube(s) in contact with at least one nucleotide sequence and optionally with a spacer as previously defined.

Advantageously, the reactive entity formed on the surface of a CNT during step (i) has
a group chosen from the group consisting of a carboxyl function, an aryl group of the polyaromatic aryl type, a radical entity, a hydroxyl function, an alcohol function, an amine function, an ester function, an aldehyde function, a hydrazide function, a ketone function, an epoxy function, an isocyanate function, a maleimide function, a diene and a thiol function or
an alkyl group as previously defined, substituted by such a group.

Concerning step (i), reviews exist in the literature on the covalent functionalization of CNTs [13].

Several examples will be noted here of methods that can be implemented during step (i) because they generate reactive entities on the CNT surface and make it possible to graft biomolecules such as nucleotide sequences afterwards:
oxidation of the CNTs using strong oxidants such as HNO₃/H₂SO₄ [14], H₂O₂ [15], KMnO₄/H₂SO₄ [16], etc. Such a liquid-phase oxidation makes it possible to generate oxygenated functions such as alcohol, carboxyl and ketone functions on the ends or defects of the CNTs;
arylation of the CNTs by diazonium [17];
functionalization of the CNTs by 13-dipolar cycloaddition [18];
functionalization of the CNTs by cycloaddition [2+1] [19].

These CNTs carrying one (or more) reactive entity(ies) as previously defined can then react directly with one (or more) nucleotide sequence(s) or with one (or more) spacer(s) as previously defined. During step (ii) and in the case of a reaction involving a spacer, the latter can react with the nucleotide sequence, before, during or after the reaction of said spacer with the CNT.

In a second alternative of the method according to the present invention, during the functionalization of the CNT(s) by one (or more) nucleotide sequence(s) in the context of the method according to the invention and in particular in the context of steps (a₁) and (a₂), the nucleotide sequence non-covalently binds to said nanotube(s). In fact, several nucleotide sequences, identical or different, can non-covalently bind to a same CNT.

Owing to the hydrophobic aromatic surface of the CNTs, the latter can be functionalized by nucleotide sequences non-covalently outside or inside the CNTs. For example, one (or more) nucleotide sequence(s) can penetrate the cavity of an open MWNT, and the transport of this (or these) nucleotide sequence(s) inside the MWNT can be followed by fluorescence [20]. The immobilization of the nucleotide sequences on the CNTs then occurs through hydrophobic interactions and Van der Waals interactions, the hydrophobic interaction playing a more important role.

The advantage of a non-covalent functionalization lies in the fact that it keeps the structure of the CNTs. It is thus possible to compare the properties of the CNTs before and after functionalization, such as the electronic and spectroscopic properties, which can be important for certain applications.

Advantageously, this non-covalent bond can be indirect. In fact, the functionalization of the CNTs by one (or more) nucleotide sequence(s) can be done via an intermediate molecule capable of non-covalently bonding to said CNT and bonding, covalently or non-covalently, to said nucleotide sequence. More particularly, said intermediate molecule bonds covalently to said nucleotide sequence via a group chosen from the group consisting of a carboxyl function, an aryl group, a radical entity, a hydroxyl function, an alcohol function, an amine function, an ester function, an aldehyde function, a hydrazide function, a ketone function, an epoxy function, an isocyanate function, a maleimide function, a diene and a thiol function or via an alkyl group as previously defined, substituted by such a group.

Indeed, it is known that pyrene strongly adsorbs on the surface of the CNTs by “π-π stacking.” Examples of intermediate molecules therefore include a pyrene derivative having at least one group as previously defined and, in particular, the ester of 1-pyrenebutyric acid and N-hydroxysuccinimide capable of reacting with an amine function borne by said nucleotide sequence.

In the context of the second embodiment of the present invention, step (b₂) consists in eliminating any nucleotide sequence not having functionalized a CNT to prevent “false” positives due to free nucleotide sequences. One skilled in the art knows different techniques that can be used for this elimination. Advantageously, step (b₂) can comprise at least one wash, at least one centrifugation and/or at least one filtration, or any other means available to one skilled in the art in his common practice, such as dialysis or ultrafiltration. One skilled in the art will be able to choose a suitable buffer for the wash(es) according to the type of functionalization done (covalent or non-covalent). This buffer can for example be water or PBS.

The methods for amplifying a nucleotide sequence are well known today and are based on the use of an enzyme of the polymerase type that has the property of accurately copying a genetic sequence of a polynucleotide from a primer zone (double helix zone). Consequently, in the context of the present invention, the amplification done in steps (a), (c₁) or (c₂) can also be an amplification by a traditional polymerase chain reaction (PCR) as well as any PCR alternative known by those skilled in the art such as an asymmetrical PCR, an interlaced thermal asymmetrical PCR, a temperature-gradient PCR, an endpoint PCR, a multiplex PCR, a real-time (or quantitative) PCR, RT-PCR (Reverse Transcription-Polymerase Chain Reaction), a multiplex RT-PCR, a NASBA (Nucleic Acid Sequence Based Amplification), an LCR (Ligase Chain Reaction) or a TMA (Transcription Mediated Amplification). The amplification method used in the context of the present invention can also comprise improvements such as the “hot start” or “touchdown” techniques or the use of dUTP in place of dTTP for decontamination by UNG (Uracil-DNA glycosylase) of any new sample.

“RT-PCR” refers to reverse transcription followed by a polymerase chain amplification. An RT-PCR therefore includes two steps: a reverse transcription step, i.e. for synthesis of a single-helix DNA complementary to an RNA sequence, implementing a reverse transcriptase followed by a polymerase chain amplification step.

“Multiplex PCR” refers to an amplification method aiming to amplify more than one amplicon at a time. This technique uses a set of pairs of amplification primers, each pair of primers being designed or adapted to amplify a different nucleotide sequence.

“Multiplex RT-PCR” refers to a multiplex PCR as previously defined preceded by a reverse transcriptase as previously defined.

“Real-time PCR” refers to an amplification method that makes it possible to detect and/or quantify the presence of the amplicons during the PCR cycles, in particular owing to a fluorescent marker. The increase in the amplicons or the signal related to the quantity of amplicons formed during the PCR cycles is used to detect and/or quantify a given nucleotide sequence in the solution subjected to the PCR.

NASBA refers to an RNA amplification method without changing temperature by using a promoter primer for the T7-RNA polymerase, a reverse transcriptase, dNTPs (desoxyribonucleotide triphosphates) and NTPs (ribonucleotide triphosphates), as well as an RNAse-H.

“LCR” refers to an amplification method using a DNA ligase and two complementary primers of the target and adjacent DNA that are bound together by DNA ligase.

“TMA” refers to an isothermal RNA amplification method requiring two enzymes that are a reverse transcriptase and a RNA polymerase and two primers with a primer containing a promoter sequence for the RNA polymerase and the other primer capable of bonding to the neosynthesized DNA sequence.

It should be stressed that the amplification steps (a), (c₁) and (c₂) are carried out in the presence of any CNTs that may be present.

The conditions implemented in steps (a), (c₁) and (c₂) in particular comprise the presence of a reactive medium and temperature conditions.

The reactive mixture used during steps (a), (c₁) and (c₂) can be any mixture of reagents for PCR or any PCR alternative which is commercially available, such as the kits sold by the companies Roche Applied Science or Applied Biosystems.

Alternatively, the reactive mixture used during the amplification steps (a), (c₁) and (c₂) can have any composition from among all of the reactive mixtures described in the state of the art for PCR or any PCR alternative. One skilled in the art will know how to prepare such a reactive mixture depending on the type of PCR done in steps (a), (c₁) and (c₂) according to the invention.

The reactive mixture comprises one (or more) element(s) chosen from at least one enzyme chosen from the group consisting of a DNA or RNA polymerase such as the Taq polymerase or the T7-RNA polymerase, a reverse transcriptase, a RNAse-H and a DNA ligase; a salt such as TRIS (for trishydroxymethylaminomethane), KCl, NaCl or MgCl₂; deoxyribonucleotide triphosphates, possibly marked, such as dATP, dGTP, dTTP, dCTP and possibly dUTP; ribonucleotide triphosphates, possibly marked, such as ATP, GTP, TTP, UTP, CTP; at least one pair of primers, either specific or degenerate, which can comprise from 10 to 100 base pairs, in particular from 15 to 50 base pairs and, in particular, from 15 to 35 base pairs; and an oligo-dT or specific primer in particular useful for reverse transcriptase or for the T7-RNA polymerase.

Furthermore, the reactive mixture can contain other additives, and in particular additives known to improve the effectiveness of PCR such as BSA (Bovine Serum Albumin), betaine, formamide, dimethyl sulfoxide, gelatin, glycerol, spermidine, ammonium sulfate, Acetamide or Amide C2, Tween-20, polyethylene glycol (PEG) 6000, proteins such as “Single Strand binding protein from E. Coli” marketed by Sigma Aldrich (increased specificity of the reaction), or the “T4 Gene32 Protein from E. Coli B infected with phage T4am134/amBL292/amE219” marketed by Roche Applied Science (increase in the production output of long PCR products, or the output of the reaction in the presence of inhibitors such as humic acid), RNase inhibitors such as “Ribonuclease Inhibitor” or Diethyl pyrocarbonate, which improve the output of the reverse transcription during a RT-PCR.

This reaction mixture can contain, when the amplification of steps (a), (c₁) and (c₂) is a multiplex

RT-PCR or a multiplex PCR, 2 to 100 different pairs of primers, each pair of primers being specific or degenerate and being able to comprise 10 to 100 base pairs, in particular 15 to 50 base pairs and, more particularly, 15 to 35 base pairs.

An example of a reaction mixture that can be used for PCR amplification during steps (a), (c₁) and (c₂) of the invention comprises between 50 and 100 mM of Tris, between 10 and 100 mM and, advantageously, 50 mM of KCl (or NaCl), between 1 and 5 mM of MgCl₂, between 20 μM and 1 mM of a dNTP mixture containing dCTP, dATP, dTTP, dGTP and possibly dUTP, between 0.01 and 0.2 U/μl of Taq Polymerase either hot start or not, primers comprising 10 to 100 base pairs, in particular 15 to 50 base pairs, and, more particularly, 15 to 35 base pairs and which can be specific or degenerate. Each primer is advantageously present, in this reactive mixture, at a concentration between 1 and 200 nM and, in particular, between 10 and 100 nM.

If the amplification in steps (a), (c₁) and (c₂) uses a reverse transcription, the latter can last from 5 to 90 min, typically 30 min, and is done at a temperature between 25 and 60° C.

When the amplification done in steps (a), (c₁) and (c₂) is not an isothermal amplification, thermal cycles are necessary. All temperature and duration conditions for the different cycles (denaturation, hybridization and elongation), known by those skilled in the art and appropriate to the type of

PCR used during these steps, are usable.

Thus, the amplification during steps (a), (c₁) and (c₂) and in particular the PCR can:
comprise two temperatures with a hybridization-elongation plateau between 50 and 70° C., advantageously 60° C., maintained for 5 to 300 seconds and a denaturation plateau at 95° C. maintained for 1 to 30 seconds;
comprise three temperatures with a hybridization level between 50 and 65° C., advantageously 60° C., maintained for 5 to 300 seconds, an elongation plateau between 65° C. and 75° C., advantageously 72° C., maintained for 5 to 300 seconds and a denaturation plateau at 95° C. maintained for 1 to 30 seconds;
or be “touch down” cycles consisting of gradually decreasing the hybridization temperature by 1° C. per cycle, for example.

The reaction during steps (a), (c₁) and (c₂) may be conducted in standard commercially-available plastic tubes and in particular with any thermocycler. The reaction volumes in this case are between 5 and 100 μl.

The reaction in steps (a), (c₁) an d(c₂) may be conducted using a microsystem adapted for PCR or RT-PCR such as a “PCR chip.” In this case, the reaction volumes are much smaller and typically between 10 nl and 1 μl. Moreover, such microsystems have the advantage of being very small and adapted for the development of portable analysis systems usable directly on the implementation site of the method according to the invention, such as a CNT production site or a research laboratory [21].

A pre-amplification of the sample in a larger volume on the microsystem or not can be done if necessary to improve the detection limit of the method.

In this case, approximately ten PCR cycles can be done for this pre-amplification step. It can be done in monoplex mode, i.e. with a single pair of primers per reaction mixture and by performing as many reactions in parallel as there are pairs of primers tested. It may also be done in multiplex mode, i.e. with all or some of the primers tested in the same reaction mixture. In this last mode, modifications of the PCR protocol in terms of concentration of primers, time and temperature of the hybridization-elongation plateaus may be done because it has been shown that it is thus possible to perform this pre-amplification step while preserving the concentration ratio of the different genic matrices. It has also been shown that this method is effective for a very large number of primer sequences without sequence optimization for the multiplex amplification.

The detection of any amplified product(s) during steps (a), (c₁) and (c₂) can be done, during steps (b), (d₁) and (d₂), using all of the methods adapted to detecting an amplification product.

Examples of methods usable in steps (b), (d₁) and (d₂) include:
methods of the electrophoresis type, which make it possible to detect the amplification product after migration in a gel or any other adapted matrix, the migration distance being connected to the size of said product;
solid surface hybridization methods; and/or
real-time PCR.

These different methods may potentially be used successively. They will be used while being based on the implementation methods that are known and described in the state of the art.

In the case of solid surface hybridization, the reactive mixture obtained after steps (a), (c₁) and (c₂) and potentially containing at least one amplification product is brought into contact with a device whereof the surface has previously been functionalized with oligonucleotides with a known sequence. The surface can be functionalized with oligonucleotides having different sequences by localizing each deposit in the form of spots, for example. Commercial devices such as Affymetrix chips or Agilent chips in particular are usable. The traditional solid surface oligonucleotide deposition techniques (“spotting” with a robot of the oligonucleotides on functionalized surface or in situ synthesis) are adapted to the preparation of hybridization detection devices.

The reaction mixture obtained after steps (a), (c₁) and (c₂) and potentially containing at least one amplification product can be deposited either without modification on the device, or by adding a solution making it possible to optimize the reactive mixture for hybridization in terms of selectivity relative to the sequence of oligonucleotides and the hybridization kinetics. This optimized buffer can contain one (or more) species known in the state of the art and selected from amongst the monovalent salts such as, for example, NaCl or KCl; one (or more) species making it possible to buffer the pH of the solution; additives making it possible to reduce the non-specific adsorption of oligonucleotides sought or marked with a fluorophore such as exogenous DNA; proteins known to saturate the surfaces by adsorption; and additives known to accelerate the hybridization reaction such as known agents condensing the DNA such as PEG, multivalent ions-spermine, spermidine, magnesium, cobalt hexamine, etc. At the end of this hybridization reaction, any element that has not hybridized with the oligonucleotides fixed on the surface is eliminated by a rinsing step.

Different methods can then be used to detect whether hybridization has taken place between certain oligonucleotides attached on the surface and an amplification product obtained in solution after steps (a), (c₁) and (c₂). It is possible to use marked primers and/or marked dNTP during the amplification of steps (a), (c₁) and (c₂). The markers can contain detectable species such as radioactive or fluorescent molecules. In that case, the detection is done directly after the hybridization and rinsing phases. Also, during the amplification, certain dNTP or NTP bear a function (ex: biotin), which may be recognized by an enzyme or a conjugated protein-enzyme. Once the recognition is done, the enzyme catalyzes the transformation of the substrate into a product and one detects the product through optical or electric means. It is also possible to detect the hybridization directly using electric methods if the device functionalized for the hybridization allows it. It is also possible to proceed with a second step after hybridization—washing for detection, such as a secondary hybridization or a reaction with a substrate related to the amplification product obtained after steps (a), (c₁) and (c₂). The detection of the substrate can then be done by enzymatic reactions. Thus said secondary hybridization can be done with a detection probe bearing an enzyme and one provides a substrate to that enzyme. The enzyme catalyzes the transformation of the substrate into a product and one detects the product using optical or electric means. The method advantageously employed is the use of marked primers and/or marked dNTP, marked with fluorescent molecules, and detection by fluorescence of the hybridization product.

When real-time PCR is used, steps (a) and (b), (c₁) and (d₁) or (c₂) and (d₂) are done simultaneously or quasi-simultaneously, since the detection and optional quantification can be done upon each thermal cycle, i.e. during the amplification or at the end-point, i.e. after the last thermal cycle, therefore after the amplification.

In the case of real-time PCR, at least one specific marked probe of the amplification product able to be obtained after step (a), (c₁) or (c₂) may be added to the reactive medium.

“Marked probe” refers, in the context of the present invention, to a fluorescent probe that can attach either onto the double-helix DNA (SYBR technology with an intercalant) or onto a specific DNA sequence (Taqman and beacon technology). The specific marked probes can be any sort among the probes known and described in the state of the art. Examples of specific marked probes usable in the context of the present invention include TaqMan probes, molecular beacons, Scorpion probes and LNA probes, and any other probe making it possible to perform real-time PCR. The detection will be done by fluorescence measurement upon each thermal cycle or at the endpoint, i.e. after the last thermal cycle.

The choice between the different detection methods will be made as a function of the sought information. When little information is sought, for example when it is necessary to simultaneously test between ten and one hundred combinations of sequences, real-time PCR is particularly suitable.

However, when the number of combinations of sequences being sought is high, up to several tens of thousands, PCR followed by chip hybridization is preferable. Successive screenings may be done. For example, a PCR with non-specific detection using an intercalating agent of the double-helix DNA can be done initially: it makes it possible to reveal a double helix structure involving a primer of the reactive mixture and a nucleotide sequence present in the sample. If this non-specific detection is positive, a second analysis can be done by using real-time PCR and/or DNA chip hybridization to know which nucleotide sequence(s) is(are) present in the sample.

When the quantification of the functionalized CNTs is desired during steps (b), (d0 and (d₂), it is necessary to simultaneously carry out a real-time PCR with a range of oligonucleotides with known concentrations. The concentration of oligonucleotides in the tested sample can thus be deduced from the reference range. The marking rate of the CNTs also being known, the concentration of CNTs in the tested sample can thus be determined.

The present invention lastly relates to the use of a kit of elements comprising:
at least one element chosen from at least one enzyme such as a thermostable DNA or RNA polymerase such as Taq polymerase or T7-RNA polymerase, a reverse transcriptase, an RNase-H and a DNA ligase; a salt such as TRIS (for trishydroxymethylaminomethane), KCl, NaCl or MgCl₂; deoxyribonucleotide triphosphates, potentially marked, such as dATP, dGTP, dTTP, dCTP and possibly dUTP; ribonucleotide triphosphates, possibly marked, such as ATP, CTP, TTP, UTP and GTP; at least one pair of primers, possibly marked, specific or degenerate, which can comprise 10 to 100 base pairs, in particular 15 to 50 base pairs and, more particularly, 15 to 35 base pairs; an oligo-dT or specific primer, in particular useful for reverse transcription or for T7-RNA polymerase and a marked probe;
optionally at least one nucleotide sequence, and
to detect, identify and optionally quantify at least one carbon nanotube in a sample.

Said kit can also comprise one (or more) additive(s) as previously described and/or one (or more) device(s) and microdevice(s) as previously described.

Other features and advantages of the present invention will appear to one skilled in the art upon reading the examples below provided as an illustration and not as a limitation, with reference to the appended figures.

### BRIEF DESCRIPTION OF THE DRAWINGS

FIG. 1 proposes a diagram representative of the functionalization of the CNTs by the DNA.

FIG. 2 presents the fluorescence during the real-time PCR in a drop of 65 nl on a chip operating by electrowetting (curve with diamonds) and in a traditional plastic tube for real-time PCR with 10 μl of solution (curve with squares).

FIG. 3 shows a reference curve for the quantitative detection of functionalized nanotubes with the threshold cycle (“Ct”) as a function of the concentration of functionalized nanotubes expressed in weight of nanotubes/μl.

### DETAILED DESCRIPTION OF THE INVENTION

### 1. COVALENT FUNCTIONALIZATION OF THE CNTS WITH DNA

In this example, the nanotubes implemented are multi-wall nanotubes (MWNT) provided by Arkema. The DNA has been synthesized with an amine at the 5′-terminal end.

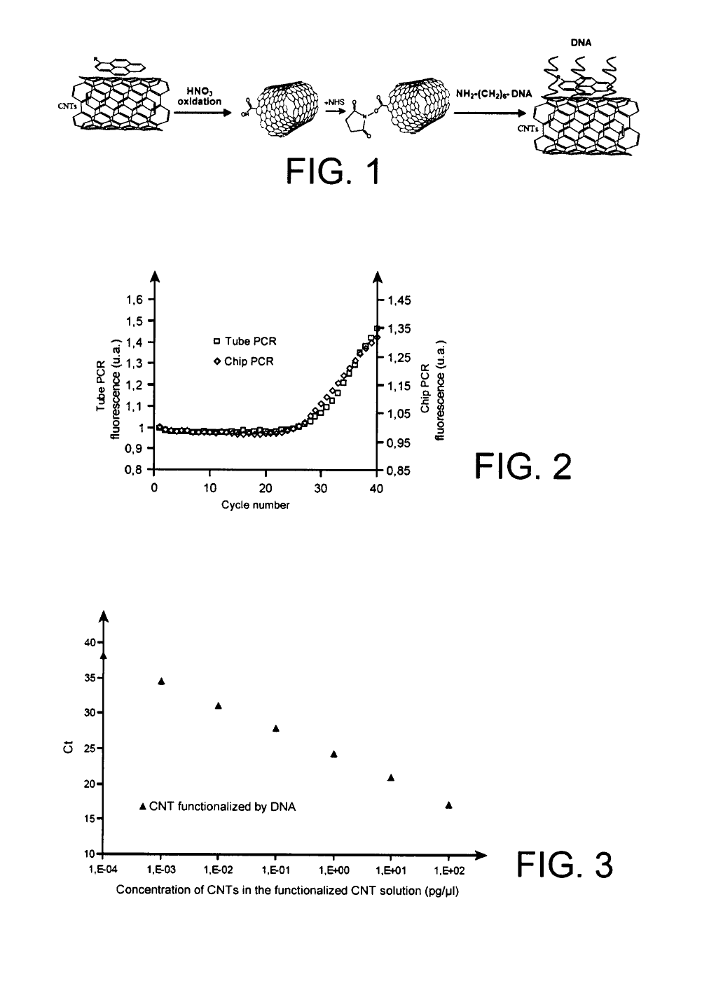

The protocol of the covalent functionalization of the MWNTs by DNA includes three steps shown in FIG. 1 and consisting in:
oxidizing the MWNTs to generate carboxylic groups on the MWNTs;
activating the carboxylic groups by N-hydroxysuccinimide (NHS);
coupling the activated carboxylic groups with the amines of DNA.

Three batches of MWNTs have been done, two of which are used to monitor the functionalization, by varying the parameters thereof:
Batch A, for which the oxidation step has been omitted;
Batch B, for which, after the oxidation step, the coupling agent (NHS and DCC) has been omitted during the activation step;
Batch C, for which the three complete steps have been carried out.

1.1. Oxidation of the Nanotubes.

Fifteen mg of MWNTs were added into 20 ml of H₂SO₄+HNO₃ (3:1). The mixture underwent ultrasound treatment for 5 h (ultrasound bath) at 30° C. The nanotube suspension was diluted in water and filtered on a membrane (0.2 μm). The MWNTs were recovered from the membrane and rinsed several times in water.

They were then treated with HCl (20 mL, 1 M) to transform the carboxylate groups borne by the MWNTs into carboxylic groups. The thereby treated MWNTs, i.e. oxidized MWNTs, were vacuum-dried at room temperature.

1.2. Synthesis of the Activated MWNTs.

Approximately 2.4 mg of oxidized MWNTs (˜0.2 mmol of C) were dispersed in 40 ml of DMF. The suspension was purged with N₂ for 30 min. NHS (46 mg, 0.4 mmol) was solubilized in 10 ml of DMF. The NHS solution was added into the suspended nanotubes with a syringe.

Then, dicyclohexyl carbodiimide (DCC) (82.4 mg, 0.4 mmol) was solubilized in 10 ml of DMF, and added into the suspended MWNTs. The mixture was stirred for 30 min at room temperature. 4-Dimethylaminopyridine (DMAP) (2.4 mg, 0.02 mmol) was solubilized in 10 ml of DMF and added, dropwise, to the suspended MWNTs. The reaction was kept at room temperature under N₂ for 18 h. After the reaction, the activated MWNTs were recovered on a filtration membrane, and rinsed with DMF. Then, they were vacuum-dried.

1.3 Coupling Activated MWNTs With DNA.

The activated MWNTs (˜2 mg) were dispersed in DMF (5 ml) by ultrasound treatment. Ten μl of oligonucleotides (76 bases, 100 μM) were diluted in 3 ml of Na₂HPO₄ (0.2 M). The oligonucleotide solution was added, dropwise, into the activated MWNT suspension under magnetic agitation.

The coupling reaction was done at room temperature for 18 h. After the reaction, the functionalized MWNTs were recovered on a filtration membrane and rinsed 3 times with DMF and 3 times with deionized water (15 ml upon each rinse).

The activated MWNTs and the functionalized MWNTs were characterized by Attenuated Total Reflection Infrared (ART-IR) and by X-ray photoelectron spectroscopy (XPS).

The ATR-IR spectrum of the activated MWNTs shows an adsorption peak corresponding to bond C═O, characteristic of the NHS groups.

The functionalized MWNTs, i.e. coupled with the DNA, were characterized by XPS. The percentage of phosphorus element, which does not exist in the activated MWNTs, is 0.23%. This percentage corresponds to a grafting output of 10 to 100 oligonucleotides per MWNT according to the size of the MWNTs.

### 2. DETECTION OF FUNCTIONALIZED CNTS WITH AN OLIGONUCLEOTIDE BY PCR ON MICROSYSTEM

Real-time PCR detection was done on a microsystem making it possible to manipulate very small drops (65 nl).

The operation of the microsystem is based on the principle of electrowetting and makes it possible to carry out the following unitary micro-fluidic steps: formation of drops with a known volume and that are reproducible, movement of the drops, mixture of two drops of solution that may be different with homogenization in several seconds [21].

During the PCR done with a functionalized CNT solution with oligonucleotides, the entire chip was heated during the PCR thermal cycles.

A PCR with a functionalized CNT suspension with oligonucleotides was done on the chip. The sequence of the oligonucleotide grafted on the CNTs is 5′-NH2-C6-TTTTTCGGGTAACGTCAATGAGCAAAAAAATATCATTGGTGTCGG ATACCCAAGGAGCATGTATTAGGCACGCCGC-3′ (SEQ ID NO. 1 in the appended sequence list). The sequences of the primers are CGGGTAACGTCAATG AGCAAA (primer forward, SEQ ID NO. in the appended list of sequences) and GCGGCGTGCCTAATACATGC (primer reverse, SEQ ID NO. 3 in the appended list of sequences) and that of the probe: 5-FAM-CACCAATGATATTTT-MGB-3′ (SEQ ID NO. 4 in the appended list of sequences). The reaction mixture contains the buffer for the AmpliTaq Gold enzyme without MgCl₂1× (ABI), BSA at 0.8 mg/mL, MgCl₂ at 3 mM, nucleotides (dATP, dTTP, dCTP, dGTP) at 200 μM, betaine at 450 mM and AmpliTaq gold 0.5 U/μl (ABI).

The sample is first mixed in a tube with the PCR reagents for the first step for denaturation and activation of the Taq Polymerase (10 min at 95° C.). This solution is then introduced onto the chip through a hole in the cap and by activating the electrodes. A drop of 65 nl of said solution is then formed by activating the electrodes, then the thermal cycles are carried out (95° C. 10 sec, 60° C. 20 sec) while heating the entire chip with a Peltier element in contact with the chip. The reading of the fluorescence of the drop is done at the end of each thermal level at 60° C. with an optical microscope.

In parallel, 10 μl of the same initial reaction mixture (sample+reagents for the PCR) was placed in a reaction tube for the PCR and it was done with a commercial PCR apparatus in real time (Stratagene Mx3005P) with thermal cycles of 60° C. 60 sec, 95° C. 60 sec.

To finish, the fluorescence signal obtained in the 65 nl drop was compared to the signal obtained in 10 μl of solution in a traditional plastic tube with a commercial PCR apparatus in real time (MX3005P Stratagene). It appears that the normalized signal obtained in both cases is absolutely similar (FIG. 2).

### 3. EXAMPLE OF FUNCTIONALIZED CNT QUANTIFICATION BY TUBE PCR

A PCR was done with successive dilutions of marked CNTs with an oligonucleotide and prepared according to point 1 above.

The quantity of CNTs initially used for the marking is known and it is therefore possible to connect each cycle threshold (Ct) obtained during said PCR with the CNT concentration in the dispersion during the PCR. It is thus possible to establish standard curves, like that shown in FIG. 3, making it possible to then yield a quantitative result after analysis of an unknown sample by linking the obtained Ct with the CNT concentration.

Based on the reference curve of FIG. 3, a new sample yielding a Ct of 31 contains 10⁻³ pg/μl of functionalized nanotubes.

### REFERENCES

[1] Sayes and coll., 2006, <<Functionalization density dependence of single-walled carbon nanotubes cytotoxicity in vitro>>, Toxicology Letters, vol. 161, p. 135-142.

[2] Wörle-Knirsch and coll., 2006, <<Oops they did it again! Carbon nanotubes hoax scientists in viability assays>>, Nano Letters, vol. 6, p. 1261-1268.

[3] Lam and coll., 2004, <<Pulmonary toxicity of single-wall carbon nanotubes in mice 7 and days after intratracheal instillation>>, Toxicological Science, vol. 77, p. 126-134.

[4] Roberts and coll., 2007, <<In vivo biomodification of lipid-coated carbon nanotubes by daphnia magna>>, Environ. Sci. Technol., vol. 4, p. 3025-3029.

[5] Kostarelos, 2008, <<The long and short of carbon nanotube toxicity>>, Nature Biotechnology, vol. 26, p. 774-776.

[6] Donaldson and coll., 2006, <<Carbon nanotubes: a review of their properties in relation to pulmonary toxicology and workplace safety>>, Toxicological Sciences, vol. 92, p. 5-22.

[7] Tantra and coll., 2007, <<The detection of airborne carbon nanotubes in relation to toxicology and workplace safety>>, Nanotoxicology, vol. 1, p. 251-265.

[8] Leeuw and coll., 2007, <<Single-walled carbon nanotubes in the intact organism: near-IR imaging and biocompatibility studies in Drosophila>>, Nano Letters, vol. 7, p. 2650-2654.

[9] Williams and coll., 2002, <<Carbon nanotubes with DNA recognition>>, Nature, vol. 420, p. 761.

[10] Lee and coll., 2004, <<Electrically addressable biomolecular functionalization of carbon nanotubes and carbon nanofiber electrodes>>, Nano Lett., vol. 4, p. 1713-1716.

[11] International application WO 2004/000727 (CEA) published on Dec. 31, 2003.

[12] International application WO 03/053846 (Universities Space Research Association) published Jul. 3, 2003.

[13] Bahr and coll., 2002, <<Covalent chemistry of single-wall carbon nanotubes>>, J. Mater. Chem., vol. 12, p. 1952-1958.

[14] Liu and coll., 1998, <<Fullerene pipes>>, Science, vol. 280, p. 1253.

[15] Miyata and coll., 2006, <<Selective oxidation of semiconducting single-wall carbon nanotubes by hydrogen peroxide>>, Science, vol. 110, p. 25..

[16] Hwang and coll., 1995, <<Efficient cleavage of carbon graphene layers by oxidants>>, Chem. Commu., vol. 2, p. 173.

[17] Marcoux and coll., 2004, <<Electrochemical functionalization of nanotube films: growth of aryl chain on single-walled carbon nanotubes>>, New J Chem., vol. 28, p. 302.

[18] Wang and coll., 2005, <<Microwave-induced rapid chemical functionalization of single-walled carbon nanotubes>>, Carbon, vol. 43, p. 1015.

[19] Hu and coll., 2003, <<Sidewall functionalisation of single-walled carbon nanotubes by addition of dichlorocarbene>>, J Am. Chem. Soc., vol. 125, p. 14893.

[20] Ito and coll., 2003, <<Observation of DNA transport through a single carbon nanotube channel using fluorescence microscopy>>, Chem. Commun., p. 1482-1483.

[21] Fouillet and coll., 2008, <<Digital microfluidic design and optimization of classic and new fluidic functions for lab on a chip systems>>, Microfluidics and Nanofluidics, vol. 4, p. 159-165.

1.-16. (canceled)

17. A method for detecting, optionally identifying, and optionally quantifying, the presence of at least one carbon nanotube in a sample, the method comprising:
(a) subjecting the sample to conditions enabling amplification of a nucleotide sequence with at least one primer capable of amplifying the nucleotide sequence, to obtain a first product; and
(b) detecting, optionally identifying, and optionally quantifying, an amplification product optionally present in the first product from (a), wherein the optionally present carbon nanotube has been functionalized by the nucleotide sequence prior to the subjecting (a).

18. The method of claim 17, comprising:
a₁) preparing at least one carbon nanotube functionalized by at least one nucleotide sequence;
b₁) taking a sample that optionally comprises carbon nanotube;
c₁) subjecting the sample to conditions enabling the amplification of the nucleotide sequence with at least one primer capable of amplifying the nucleotide sequence, to obtain the first product; and
d₁) detecting, optionally identifying, and optionally quantifying, an amplification product optionally present in the first product after (c₁).

19. The method of claim 17, comprising:
at least one selected from the group consisting of isolating any nanotube present in the sample and purifying any nanotube present in the sample, to obtain a second;
a₂) contacting the second nanotube with at least one nucleotide sequence under conditions enabling functionalization of the second nanotube by the nucleotide sequence;
b₂) eliminating any nucleotide sequence not involved in the functionalization;
c₂) subjecting the sample to conditions enabling the amplification of the nucleotide sequence with the primer capable of amplifying the nucleotide sequence, to obtain the first product; and
d₂) detecting and optionally quantifying an amplification product optionally present in the product after (c₂).

20. The method of claim 17, wherein the sample comprises at least one selected from the group consisting of a biological sample, a sample of city water, a sample of river water, a sample of sea water, a sample of lake water, a sample of ground water, a sample of air-cooled tower water, an aerial sample, a ground sample, and a sample obtained on an industrial site.

21. The method of claim 17, wherein the nucleotide sequence is selected from the group consisting of an oligonucleotide, a modified oligonucleotide, a deoxyribonucleic acid (DNA), a modified DNA, a ribonucleic acid (RNA), and a modified RNA,
or a portion or fragment thereof.

22. The method of claim 17, wherein, during the functionalization of the at least one nanotube by the nucleotide sequence, the nucleotide sequence bonds covalently to the nanotube.

23. The method of claim 17, wherein, during the functionalization of the nanotube by the nucleotide sequence, the nucleotide sequence bonds covalently and indirectly to the nanotube.

24. The method of claim 23, wherein the covalent and indirect bond is done via a spacer comprising a first and a second distinct chemical function and capable of forming a covalent bond,
wherein the first distinct chemical function comprises a group carried by the carbon nanotube, and
wherein the second chemical function comprises a group carried by the nucleotide sequence.

25. The method of claim 24, wherein the chemical functions, which are identical or different, are selected from the group consisting of a carboxyl function, an aryl group, a radical entity, a hydroxyl function, an alcohol function, an amine function, an ester function, an aldehyde function, a hydrazide function, a ketone function, an epoxy function, an isocyanate function, a maleimide function, a diene, and a thiol function.

26. The method of claim 22, wherein the functionalization comprises:
(i) subjecting the sample or nanotube to conditions enabling at least one reactive entity to be formed on a surface of a carbon nanotube; then
(ii) contacting the sample or nanotube with at least one nucleotide sequence and optionally with a spacer comprising a first and a second distinct chemical function and capable of forming a covalent bond,
wherein the first distinct chemical function comprises a group carried by the carbon nanotube, and
wherein the second chemical function comprises a group carried by the nucleotide sequence.

27. The method of claim 26, wherein the reactive entity formed on the surface of the carbon nanotube during (i) comprises:
a moiety selected from the group consisting of a carboxyl function, an aryl group, a radical entity, a hydroxyl function, an alcohol function, an amine function, an ester function, an aldehyde function, a hydrazide function, a ketone function, an epoxy function, an isocyanate function, a maleimide function, a diene, and a thiol function, or
an alkyl group substituted by the moiety.

28. The method of claim 17, wherein, during the functionalization of the nanotube by the nucleotide sequence, the nucleotide sequence bonds non-covalently to the nanotube.

29. The method of claim 28, wherein, during the functionalization of the nanotube by the nucleotide sequence, the nucleotide sequence bonds non-covalently and indirectly to the nanotube.

30. The method of claim 29, wherein the non-covalent and indirect bond is done via an intermediate molecule capable of bonding non-covalently to the carbon nanotube and of bonding, covalently or non-covalently, to the nucleotide sequence.

31. The method of claim 17, wherein the amplification comprises
a Polymerase Chain Reaction (PCR) amplification,
an asymmetrical PCR,
an interlaced thermal asymmetrical PCR,
a temperature gradient PCR,
an endpoint PCR,
a multiplex PCR,
a real-time PCR,
an RT-PCR (Reverse Transcription - Polymerase Chain Reaction),
a multiplex RT-PCR,
a NASBA (Nucleic Acid Sequence Based Amplification),
an LCR (Ligase Chain Reaction), or
a TMA (Transcription Mediated Amplification).

32. A method of manufacturing a kit of elements, the method comprising combining:
at least one element selected from the group consisting of an enzyme, an optionally marked deoxyribonucleotide triphosphate, an optionally marked ribonucleotide triphosphate, a pair of optionally marked primers which are specific or degenerate, an oligo-dT primer, a specific primer, and a marked probe; and
at least one nucleotide sequence, adapted to detect, identify, and optionally quantify at least one carbon nanotube in a sample,
with the kit.

33. The method of claim 32, wherein the enzyme is present and comprises thermostable DNA or RNA polymerase, Taq polymerase, T7-RNA polymerase, a reverse transcriptase, an RNase-H, or a DNA ligase

34. The method of claim 32, wherein the triphosphate is present and comprises at least one selected from the group consisting of dATP, dGTP, dTTP, dCTP, dUTP, ATP, CTP, TTP, UTP, and GTP.

35. The method of claim 17, comprising identifying the amplification product after (c₁).

36. The method of claim 17, wherein the enzyme is present and comprises quantifying the amplification product after (c₁).


SEQUENCE LISTING (4 sequences)

SEQ ID NO: 1 - DNA, 76 bp, Artificial Sequence
  Description of Artificial Sequence Synthetic oligonucleotide
tttttcgggtaacgtcaatgagcaaaaaaatatcattggtgtcggatacccaaggagcatgtattaggcacgccgc

SEQ ID NO: 2 - DNA, 21 bp, Artificial Sequence
  Description of Artificial Sequence Synthetic forward primer
cgggtaacgtcaatgagcaaa

SEQ ID NO: 3 - DNA, 20 bp, Artificial Sequence
  Description of Artificial Sequence Synthetic reverse primer
gcggcgtgcctaatacatgc

SEQ ID NO: 4 - DNA, 15 bp, Artificial Sequence
  Description of Artificial Sequence Synthetic marked probe
caccaatgatatttt